(19) United States
(12) Patent Application Publication
(10) Pub. No.: US 2016/0030344 A1
(43) Pub. Date: Feb. 4, 2016

(54) COMPOSITIONS INCLUDING APOLIPOPROTEIN AND METHODS USING FOCUSED ACOUSTICS FOR PREPARATION THEREOF

(71) Applicant: Covaris, Inc., Woburn, MA (US)

(72) Inventors: Carl Beckett, Harvard, MA (US); James A. Laugharn, JR., Winchester, MA (US); Srikanth Kakumanu, North Billerica, MA (US)

(73) Assignee: Covaris, Inc., Woburn, MA (US)

(21) Appl. No.: 14/817,375

(22) Filed: Aug. 4, 2015

Related U.S. Application Data
(60) Provisional application No. 62/032,713, filed on Aug. 4, 2014.

(51) Int. Cl. A61K 9/127; A61K 38/17

(52) U.S. Cl. CPC A61K 9/1277; A61K 38/1709

(57) ABSTRACT

The present disclosure relates to a composition that includes an apolipoprotein and a lipid bilayer, and methods and systems for preparing the composition. The apolipoprotein may be incorporated within at least a portion of the lipid bilayer. The lipid bilayer may form a liposome or other suitable carrier for transporting the apolipoprotein. The apolipoprotein incorporated lipid bilayer may provide a suitable delivery vehicle for the apolipoprotein to the body. Compositions of the present disclosure may be formed by exposing a mixture of an apolipoprotein and a lipid formulation to focused acoustic energy which, in some embodiments, may result in a liposome that at least partially encapsulates the apolipoprotein. In some embodiments, apolipoprotein A-V may be incorporated within a liposome, where the apolipoprotein A-V is suitably bioactive, or therapeutic, when delivered to cells and/or into the body of a patient.

### CROSS REFERENCE TO RELATED APPLICATION

This application claims the benefit of U.S. Provisional Application No. 62/032,713, filed Aug. 4, 2014, which is hereby incorporated by reference in its entirety.

### BACKGROUND

1. Field

Aspects of the present disclosure relate to compositions including apolipoprotein and the use of focused acoustic energy for preparation thereof.

2. Related Art

The occurrence of cardiovascular disease has been correlated with elevated levels of triglyceride. Apolipoproteins are macromolecular protein molecules that bind lipids (e.g., fat, cholesterol, triglyceride, other oil-soluble substances) to form lipoproteins. Apolipoproteins may, in turn, act as lipid transfer carriers that transport various lipids through the lymphatic and circulatory systems. For instance, apolipoproteins have amphipathic portions (e.g., phospholipid chains) that may surround the lipid(s) to which the apolipoprotein(s) are bound, which allow for the overall lipoprotein molecule to be soluble in water, despite the lipid portion itself being insoluble to water. This allows lipids to be carried through water-based circulation (e.g., blood, lymph). Apolipoproteins may also serve as enzyme cofactors and/or receptor ligands to regulate certain enzymes in the process of lipid metabolism.

Apolipoprotein A-V (ApoA5) is a type of apolipoprotein that is formed in the liver and secreted into the plasma. ApoA5 has been found to modulate lipoprotein lipase activity and is a determinant of triglyceride levels in plasma. For example, it has been found that a lack of normal functioning ApoA5 is a risk factor for hypertriglyceridemia, that is, overall amounts of ApoA5 in the body is inversely proportional to levels of triglyceride within the plasma.

### SUMMARY

Aspects described herein relate to compositions that incorporate an apolipoprotein (e.g., ApoA5), for example, into and/or within a space enclosed by a lipid bilayer (e.g., liposome), and systems and methods for preparation thereof. In some embodiments, the apolipoprotein may be included within a portion of the lipid bilayer itself. For example, the protein may be partially encapsulated or otherwise be integrated with the lipid bilayer. That is, portions of the apolipoprotein may be located amongst or may interrupt portions of the molecules of the lipid bilayer. In some cases, the apolipoprotein may be fully encapsulated within a lumen or cavity formed by the lipid bilayer. The lipid bilayer may form a liposome, micelle, vesicle or other suitable carrier for transporting the apolipoprotein.

Compositions that include an apolipoprotein incorporated with a lipid bilayer may provide a desirable delivery vehicle for the apolipoprotein to the body. For example, when suitably incorporated with the lipid bilayer and delivered to an appropriate biological system (e.g., human liver), the presence of the apolipoprotein is able to induce a preferred biological response (e.g., reduction in overall concentration of triglyceride within the plasma after an applied lipid dose), without giving rise to toxicity or apoptosis in surrounding cells.

To prepare various embodiments of compositions in accordance with the present disclosure, a mixture including an apolipoprotein and a lipid formulation may be provided within a vessel. The mixture may be exposed to a focal zone of acoustic energy, resulting in the formation of a liposome or other lipid bilayer that at least partially encapsulates the apolipoprotein. In some embodiments, the focused acoustic energy may be transmitted through a wall of the vessel such that at least a portion of the mixture is exposed to the focal zone. In various embodiments, the focused acoustic energy may have a frequency of between about 100 kilohertz and about 100 megahertz and the focal zone may have a size dimension of less than about 2 centimeters.

Compositions in accordance with the present disclosure may be prepared based on various process characteristics, each of which may be appropriately controlled to suit incorporation of the apolipoprotein with the lipid bilayer. For instance, the focused acoustic energy may be generated by a transducer that is operated according to a number of parameters, in combination. Such parameters that may affect formation of the composition(s) may include, for example, duty factor, cycles per burst, duration of acoustic treatment, peak incident power (PIP), total amount energy applied to the sample, energy volume density, etc. Other process characteristics such as temperature around the sample, pressure within the treatment vessel, sample volume, amongst others, may also affect the formation of the composition(s).

In an illustrative embodiment, a method of preparing a lipid composition is provided. The method includes providing a mixture including an apolipoprotein and a lipid formulation in a vessel; transmitting focused acoustic energy having a frequency of between about 100 kilohertz and about 100 megahertz and a focal zone having a size dimension of less than about 2 centimeters through a wall of the vessel such that at least a portion of the mixture is exposed to the focal zone; and forming a liposome at least partially encapsulating the apolipoprotein by, at least in part, exposure of the mixture to the focal zone.

In another illustrative embodiment, an apolipoprotein composition is provided. The composition includes a protein including apolipoprotein A-V; and a lipid bilayer at least partially encapsulating the protein.

Various embodiments of the present disclosure provide certain advantages. Not all embodiments of the present disclosure share the same advantages and those that do may not share them under all circumstances.

Further features and advantages of the present disclosure, as well as the structure of various embodiments of the present disclosure are described in detail below with reference to the accompanying drawings.

### BRIEF DESCRIPTION OF THE DRAWINGS

Non-limiting embodiments of the present disclosure will be described by way of example with reference to the accompanying figures, which are schematic and are not intended to be drawn to scale. For purposes of clarity, not every component is labeled in every figure, nor is every component of each embodiment of the present disclosure shown where illustration is not necessary to allow those of ordinary skill in the art to understand the invention. In the figures:

FIG. 1 shows a schematic diagram of an acoustic treatment system in accordance with an illustrative embodiment; and

FIG. 2 shows microscope images of cells treated with various compositions.

### DETAILED DESCRIPTION

The present disclosure relates to compositions that include an apolipoprotein (e.g., ApoA5) incorporated with a lipid bilayer (e.g., liposome), and systems and methods of using focused acoustics to prepare such compositions. Such compositions may allow for the apolipoprotein(s) to be delivered to the body in a suitable manner, for therapeutic purposes. For example, the delivered apolipoprotein may serve to reduce overall concentrations of triglyceride safely, with minimal cellular or tissue toxicity, if at all.

Compositions described herein may be suitably bioactive; that is, delivery of the composition may result in the apolipoprotein being appropriately taken up by surrounding cells so as to result in a favorable biological response (e.g., reduced levels of triglyceride with minimal cell mortality). This is in contrast to some instances where it was observed that the delivery of an apolipoprotein without the lipid bilayer can be toxic to surrounding cells or tissue; or other instances where the apolipoprotein experienced degradation, with a loss or substantial decrease in therapeutic effect.

To prepare a composition in accordance with aspects of the present disclosure, for various embodiments, an apolipoprotein (e.g., ApoA5) and a lipid formulation (e.g., phosphocholine, phosphoglycerol) may be added together as various components of a sample mixture within a vessel. The mixture may be exposed to focused acoustic energy generated from a transducer spaced away from the vessel. For example, the transducer may be operated so as to transmit an acoustic wavetrain through a wall of the vessel such that at least a portion of the mixture is exposed to the acoustic focal zone. Upon appropriate exposure to the focused acoustic energy, a liposome or other suitable lipid bilayer structure that at least partially encapsulates the apolipoprotein may be formed.

The focused acoustic energy may have any suitable characteristics that, when used to process the sample mixture(s) described herein, results in a bioactive composition of apolipoprotein incorporated with a lipid bilayer, such as a liposome or other vesicle. In some embodiments, the focused acoustic energy has a frequency of between about 100 kilohertz and about 100 megahertz, and the focal zone may have a size dimension of less than about 2 centimeters. In some embodiments, to prepare a bioactive apolipoprotein carrying liposome, it may be preferable for the transducer to be operated at a duty factor of greater than or equal to 25% (e.g., 25%, 50%, 75%, 100%) and between 50 and 2,000 cycles per burst (e.g., 1,000 cycles per burst). In some embodiments, to prepare suitable apolipoprotein carrying liposomes, it may be particularly suitable for the acoustic energy output to the sample mixture to be greater than or equal to 2,000 J (e.g., between 2,000 J and 20,000 J). In some cases, the peak incident power (PIP) output of the focused acoustic energy may be approximately 80 W or more and the time of focused acoustic energy exposure may be at least about 30 seconds, giving rise to an energy output of approximately 2,400 J or more.

Generally described, a lipid bilayer is a thin membrane made up of two layers of lipid molecules, where the lipid molecules exhibit polarity. The cell membrane of most, if not all, living organisms includes a lipid bilayer, which provides a protective barrier for sub-cellular structures and regulates the transport of ions, proteins and other molecules into and out of the cell. Lipid bilayers often include phospholipids, which are molecules that have a hydrophilic head and one or more hydrophobic tails.

When exposed to water, phospholipids may self-assemble into a vesicle. For example, similar to the coalescing of oil droplets in water, the lipid molecules may form a spherical configuration (e.g., micelle) where the hydrophobic tails point toward one another and the hydrophilic portions point outward. In some embodiments, a lipid bilayer is formed as a liposome, which is a substantially spherical vesicle that has a cavity as its core and that may be used as a delivery vehicle for administration of pharmaceuticals, biological molecules, bioactive agents, or other suitable compositions.

In various embodiments, focused acoustic treatment systems may be used to form bioactive compositions that include an apolipoprotein and a lipid bilayer in accordance with the present disclosure. Although ultrasonics have been utilized for a variety of diagnostic, therapeutic and research purposes, the biophysical, chemical and mechanical effects are generally only empirically understood. Some conventional uses of sonic or acoustic energy in materials processing include “sonication,” which is an unrefined process of mechanical disruption involving the direct immersion of an unfocused ultrasound source emitting energy in the low kilohertz (kHz) range (e.g., 15 kHz) into a fluid suspension of the material being treated. Accordingly, the sonic energy produces inconsistent results due to the unfocused and random nature of the acoustic waves and are prone to induce sample overheating, as the energy is scattered, absorbed and/or not properly aligned with the target.

In contrast to some prior uses of sonic energy, the use of “focused acoustics” as described herein in the preparation of bioactive apolipoprotein compositions has significant benefits, including those listed in the present disclosure. Focused acoustics, generated under appropriate parameters, provide distinct benefits in that such technology allows for stable and reproducible preparation of apolipoprotein compositions, having a desired level of bioactivity, with little or no degradation or toxicity. One of the benefits of focused acoustics is that it provides for high-energy processing and preparation of apolipoprotein compositions with little or no adverse heating of the sample during acoustic processing (e.g., providing the ability to acoustically treat a sample isothermally).

Bioactive apolipoprotein compositions in accordance with the present disclosure may be processed in a contained environment, i.e., a closed system, enabling sterile non-contact operation, without risk of contamination. Focused acoustic treatment is highly scalable, e.g., via continuous flow through systems, to sample sizes having volumes larger than that of typical sample volumes held in single-use containers, such as a test tube, pipette tip or multiwall plate. Additionally, focused acoustic methods described herein may involve a simple process operation that requires a small amount of labor, and a generally low operator skill set in comparison to that required of conventional sonication or methods of applying acoustic energy to sample materials.

Focused acoustics may be used in accordance with adaptive focused acoustics (AFA) methods provided by Covaris, Inc., Woburn, Mass. Examples of such focused acoustic systems are described in U.S. Pat. Nos. 6,948,843; 6,719,449; 7,521,023; 7,687,026; and 8,459,121, U.S. Publication No. 2013/0026669, entitled “Systems and Methods for Preparing Nanocrystalline Compositions Using Focused Acoustics” and U.S. Publication No. 2013/0155802, entitled “Compositions and Methods for Preparing Nanoformulations and Systems for Nano-Delivery Using Focused Acoustics,” each of which are hereby incorporated by reference herein in their entireties. The above references are referred to herein for details regarding the construction and operation of an acoustic transducer and its control for various applications.

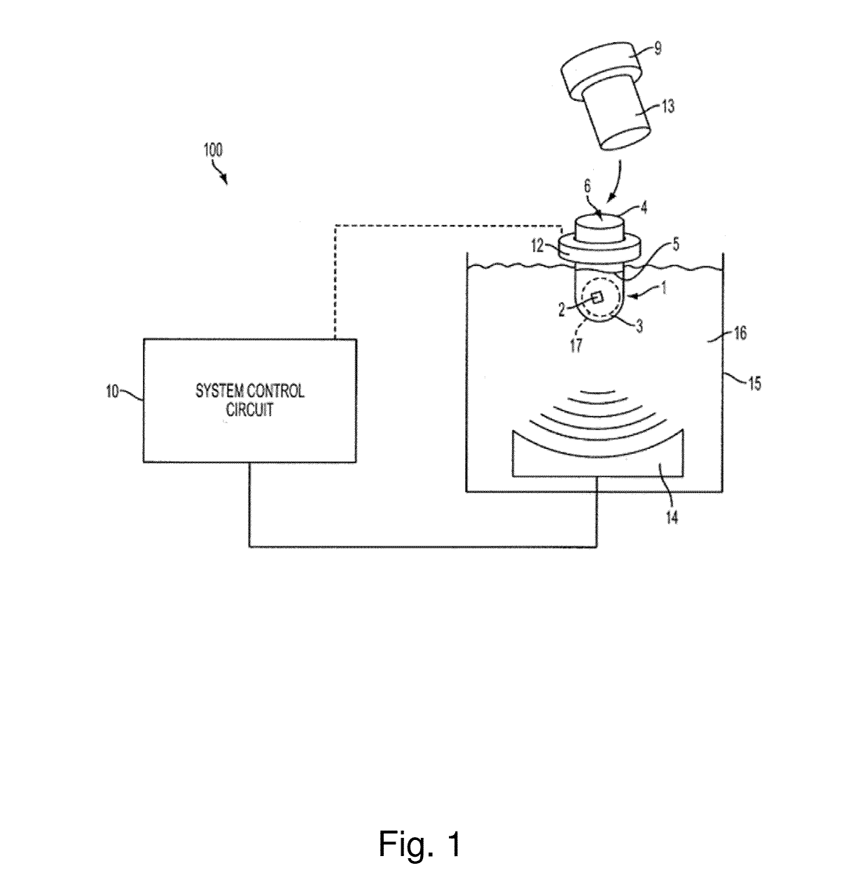

FIG. 1 shows a schematic block diagram of an acoustic treatment system 100 that incorporates one or more aspects of the embodiments described herein. It should be understood that although embodiments described herein may include most or all aspects of the present disclosure, certain aspects may be used alone or in any suitable combination with aspects of other embodiments.

In this illustrative embodiment, the acoustic treatment system 100 includes an acoustic transducer 14 (e.g., including one or more piezoelectric elements) that is capable of generating an acoustic field (e.g., at a focal zone 17) suitable to cause mixing, e.g., caused by cavitation, and/or other effects in a sample 1 contained in a vessel 4. The acoustic transducer 14 may produce acoustic energy within a frequency range of between about 100 kilohertz and about 100 megahertz such that the focal zone 17 has a width of about 2 centimeters or less. The focal zone 17 of the acoustic energy may be any suitable shape, such as spherical, ellipsoidal, rod-shaped, or column-shaped, for example, and may be positioned at the sample 1. The focal zone 17 may be larger than the sample volume, or may be smaller than the sample volume, as shown in FIG. 1.

The vessel 4 may have any suitable size or other arrangement, e.g., may be a glass tube, a plastic container, a well in a microtiter plate, a vial, or other, and may be supported at a location by a vessel holder 12. In this example, the vessel 4 is a standard rimless 13×100 mm borosilicate glass test tube, but it should be understood that the vessel 4 may have any suitable shape, size, material, or other feature, as further described herein. The vessel 4 may be formed of glass, plastic, metal, composites, and/or any suitable combinations of materials, and formed by any suitable process, such as molding, machining, extrusion, stamping, and/or a combination of processes. In some embodiments, the vessel 4 may have an interior space that is suitable to accommodate an overpressure, i.e., a pressurized state where the pressure within an interior space, or treatment area, of the vessel is greater than the pressure of the ambient environment exterior to the vessel. Various embodiments of vessels in accordance with the present disclosure are described further below.

The acoustic treatment system 100 may also include a coupling medium container 15 that is capable of holding a medium 16 (such as water or other liquid, deformable/flexible medium, gas, gel, solid, semi-solid, and/or a combination of such components) which transmits acoustic energy from the transducer 14 to the vessel 4. In embodiments where the medium 16 includes a solid or semi-solid, a container 15 need not be provided or a portion of the medium 16 itself may function as a container 15, e.g., to hold a liquid or gas portion of the medium 16. For example, in one embodiment, the transducer 14 may be attached to a solid coupling medium 16 (such as a silica material), which is also attached to a vessel holder 12, which may be formed, at least in part, by an opening or other feature of the medium 16. Thus, the transducer 14, medium 16 and holder 12 may be formed as a single integrated part, if desired.

In some embodiments, the acoustic field may be controlled, the acoustic transducer 14 may be moved, and/or the vessel 4 may be moved (e.g., by way of moving a holder 12, such as a rack, tray, platform, etc., that supports the vessel 4) so that the sample is positioned at a desired location relative to the focal zone 17. In addition, or alternatively, the transducer 14 may form the focal zone 17 so that the focal zone 17 is suitably positioned relative to the sample 1 or vessel 4.

To control the acoustic transducer 14, the acoustic treatment system 100 may include a system control circuit 10 that controls various functions of the system 100 including operation of the acoustic transducer 14 (e.g., duty factor, cycles per burst, peak incident power, duration of focused acoustic treatment, etc.) and/or other aspects (e.g., temperature around the sample, pressure within the treatment vessel, etc.). For example, the system control circuit 10 may provide control signals to a load current control circuit, which controls a load current in a winding of a transformer. Based on the load current, the transformer may output a drive signal to a matching network, which is coupled to the acoustic transducer 14 and provides suitable signal(s) for the transducer 14 to produce desired acoustic energy.

As discussed in more detail below, the system control circuit 10 may control various other acoustic treatment system 100 functions, such as positioning of the vessel 4 and/or acoustic transducer 14 (a dashed line linking the control circuit 10 to the holder 12 schematically represents an optional positioning system, e.g., including a robot, gantry, screw drive, or other arrangement to move the holder 12), receiving operator input (such as commands for system operation), outputting information (e.g., to a visible display screen, indicator lights, sample treatment status information in electronic data form, and so on), and others.

As noted above, in various embodiments, the system control circuit 10 may control other aspects of the acoustic treatment system 100, such as temperature and/or pressure of a treatment area. For example, a thermal adjustment apparatus (e.g., Peltier cooling chamber, thermal control jacket, coolant circulation system, etc.) may be provided with the coupling medium 16 and/or vessel 4, for controlling the temperature of the sample material 1 and/or coupling medium 16. For example, the temperature around the vessel holding the sample may be maintained within a suitable range, such as between 1° C. and 20° C. (e.g., between 3° C. and 10° C., or other appropriate temperature ranges). In addition, a pressure adjustment apparatus (e.g., gas feed, spring, compressive block for adjusting volume and/or applied force within a sealed arrangement, etc.) may also be provided so as to apply a suitable amount of pressure to the sample material 1 and/or coupling medium 16. In various embodiments, the control circuit 10 may monitor the temperature and/or pressure of various parts of the system (e.g., coupling medium, vessel) and adjust the appropriate parameter(s), as desired.

In this illustrative embodiment, the sample 1 includes a solid material 2 and a liquid 3, e.g., 100 milligrams of a biological sample material in 1 milliliter of distilled water. Of course, those of skill in the art will appreciate that the sample 1 is not limited to a solid material 2 in a liquid 3, as the sample 1 may take any suitable form, such as a liquid only form, a solid only form, a mixture of liquid and solid as in this embodiment, a gel, a semi-solid, a gas, and/or combinations thereof. Samples may include any suitable material(s), such as biological materials (e.g., proteins, apolipoproteins, lipid formulations, liposomes, nucleic acids, antibiotics, steroids, bioactive agents, cells, etc.).

An interface 5 separates the sample 1 from the headspace 6, which is shown to be a gaseous region immediately above the sample 1. For some power levels at the focal zone 17 and/or sample types or arrangements, acoustic energy suitable to cause mixing, e.g., lysing, extraction, permeabilizing, catalyzing, degrading, fluidization, heating, particle breakdown, sterilization, shearing and/or disruption of molecular bonds in the sample 1, may also cause portions of the sample 1 (including solid material 2 and/or liquid material 3 of the sample 1) to be splashed or otherwise ejected from the interface 5. In some cases, the ejected sample 1 may return to the main volume of sample 1, but in other cases, the ejected sample 1 may adhere to the vessel 4 above the interface 5 or otherwise fail to return to the main sample 1. In either case, the ejected sample 1 may spend a reduced amount of time in the focal zone 17.

In addition, or alternatively, acoustic energy may cause gas in the headspace 6 to be entrained into the sample 1, such as by dissolving a portion of the gas in the headspace 6 and/or by capturing bubbles of headspace gas in the sample due to motion of the liquid at the interface 5. As discussed herein, gas located within the sample 1 may interfere with acoustic energy, such as by the presence of gas bubbles or other interfacial defects at or near the focal zone 17, reflecting acoustic energy away from the sample 1 and/or by dissolved gas increasing a pressure in cavitation bubbles created by acoustic energy, thereby decreasing the rate or force at which the cavitation bubbles collapse. In some cases, gas bubbles or other interfacial defects located within the coupling medium may also interfere with the overall effectiveness of acoustic treatment.

Without wishing to be bound by theory, the collapse of cavitation bubbles may transfer significant kinetic energy to sample materials, causing the materials to be lysed, sheared, rearranged, or otherwise mechanically operated on. By increasing a pressure in such bubbles, dissolved gas in the sample may result in a reduction in the energy released by cavitation bubble collapse, reducing an effectiveness of acoustic treatment.

In accordance with some embodiments of the present disclosure, a headspace at an interface of a sample can be controlled, e.g., in volume and/or surface area presented at the interface, to reduce an amount of gas available for entrainment in the sample. Headspace size (volume and/or surface area presented at the interface 5) can be controlled in a variety of different ways. For example, a cap 9 may be engaged with the vessel 4 so as to position a headspace control member 13 near the interface 5. In this embodiment, the headspace control member 13 is attached to the cap 9 (e.g., formed as a unitary part with the cap 9), but other arrangements are possible as discussed more below. The headspace control member 13 may reduce a volume of the headspace 6 to be 50% or less (e.g., 20% or less, 10% or less, 5% or less, 2% or less, 1% or less) than the volume of the sample. In some embodiments, the volume of the headspace 6 may be 10% or less than the volume of the sample 1, even as little as 0% of the sample volume where the headspace control member 13 is in contact with the sample 1 at the interface 5.

As discussed above, focused acoustic energy may be used to prepare suitable bioactive lipid compositions incorporating apolipoprotein. Existing methods for making multilamellar and unilamellar vesicles may require sensitive biological materials to be exposed to adverse conditions, such as organic solvents, high temperatures, etc. Such methods, as known to those of skill in the art, may include detergent depletion, ethanol/ether injection, reverse-phase evaporation, freeze drying of monophase solutions, high temperature processing, injection of supercritical fluid, use of a membrane contactor, high-intensity homogenization, amongst others. The use of these methods may lead to one or more disadvantages, for example, requirements to use substantial amounts of organic solvents, uneven and/or high temperature or pressure distributions, lack of sterilization, lengthy time requirements, etc. Further, the use of some of these methods may result in loss and/or degradation of material.

Traditional sonication has been used to prepare liposomes or other vesicles, though, the unfocused and low frequency nature of traditional sonication typically leads to ineffective results. Unfocused and low frequency acoustic energy is inefficient and generally does not meet the requirements (e.g., insufficient energy provided to the composition) for producing biologically effective vesicles. For example, the use of traditional sonication may result in bioactive component of the vesicle(s) to be degraded or non-functional.

However, focused acoustic treatment in accordance with aspects of the present disclosure may be particularly effective and reproducible in forming liposomes, or other vesicles that include a lipid bilayer, having characteristics that allow for biologically active materials such as apolipoproteins to be appropriately incorporated therewith. In some cases, the use of focused acoustics to prepare certain compositions does not require the use of an organic solvent at any point in the preparation of the composition to be delivered. Though, it may be desirable to employ one or more solvents in the preparation of suitable compositions.

Focused acoustics also allows sample materials to be processed under isothermal conditions. For example, in some cases, exposure of the sample material to focused acoustic energy does not significantly raise, or lower, the temperature of the sample. That is, the acoustic energy is concentrated in a manner so as not to generate excessive heat that may otherwise adversely affect processing thereof. In some embodiments, a water bath or jacket surrounding the vessel within which the sample material is held may help to maintain the temperature of the sample during processing. Though, for various embodiments, such a water bath or jacket is not necessary to implement during focused acoustic treatment, as processing conditions may be substantially isothermal, where temperature fluctuations of the sample during treatment are low to minimal.

Bioactive compositions incorporating apolipoproteins in combination with a lipid bilayer may be prepared according to a suitable combination of process parameters, each of which may be appropriately controlled. As discussed above, such parameters may include, for example, duty factor, cycles per burst, duration of acoustic treatment, peak incident power, energy input from the transducer and/or other parameters. Other characteristics such as sample composition, sample temperature, pressure within the treatment vessel, sample volume, flow characteristics (e.g., flow rate, inlet/outlet positioning) and/or other properties may also affect overall formation of the composition(s), and its resulting properties.

The focused acoustic energy may provide energy sufficient to induce curvature in the initial lipid formulation so as to ultimately form lipid bilayer vesicles (e.g., liposomes). A number of mechanisms may give rise to the formation of lipid bilayer vesicles in accordance with the present disclosure. For example, lipid molecules may be disrupted and rearranged so as to form phospholipid bilayer fragments. When in a hydrophilic environment, to minimize the energy of a hydrophobic edge of such fragments being unfavorably exposed to hydrophilic molecules, the fragments may tend to curve into liposomes, or other vesicles. Alternatively, solubility conditions of the mixture may be manipulated, for example, by including an appropriate detergent, or other chemical, in the mixture.

Without wishing to be bound by theory, phospholipid bilayer fragments may generally be formed through bubble cavitation caused by the focused acoustics. These cavitation bubbles nucleate and grow from tiny contaminants in the surrounding liquid or other nucleation features, and increase in size as they absorb acoustic energy. As a bubble grows in size, the bubble may grow to a point where it undergoes stable cavitation, characterized by the bubble oscillating in size, yet not collapsing. Or, the bubble may undergo collapse cavitation by growing until a certain critical (i.e., resonant) size is reached, at which point the bubble expands extremely rapidly such that it is unable to stably absorb acoustic energy, causing the bubble to ultimately collapse.

During stable cavitation, the oscillating bubble may produce a radiation pressure, which causes the lipid molecules to move toward the source of the vibrations. Acoustic microstreaming around the bubble may provide shear forces that break apart the lipid into phospholipid bilayer fragments. When a phospholipid bilayer fragment forms a structure that has a radius larger than a critical radius, then the energetically favorable path for the fragment is to form a curved configuration, and ultimately form into a liposome or other vesicle. If the phospholipid bilayer fragment does not form a structure having a sufficiently large radius, it may have a tendency to fuse with other bilayer fragments until the critical radius is reached, and then self-assemble into a liposome or other appropriate vesicle, encapsulating a bioactive agent such as an apolipoprotein.

The amount of shear force around stable cavitation bubbles produced by the acoustic energy may contribute in a manner that affects the overall size of the phospholipid bilayer fragments and, consequently, overall vesicle size and distribution. The parameters of acoustic energy treatment, such as those discussed herein, may, in turn, contribute to the amount of shear force generated and, hence, dictate various characteristics of the lipid bilayer vesicle formed.

The acoustic transducer may be operated at a suitable duty factor, in combination with other parameters, to generate focused acoustic energy that leads to preferred results. As described herein, the duty factor is the percentage of time in a cycle in which the transducer is actively emitting acoustic energy. For example, a duty factor of 60% refers to the transducer being operated in an “on” state 60% of the time, and in an “off” state 40% of the time.

In some embodiments, in forming a liposome, or other vesicle, suitably incorporating an apolipoprotein therein, the acoustic transducer may be operated at a duty factor setting of greater than or equal to 10%, greater than or equal to 20%, greater than or equal to 25%, greater than or equal to 30%, greater than or equal to 40%, greater than or equal to 50%, greater than or equal to 60%, greater than or equal to 70%, greater than or equal to 75%, or greater than or equal to 80%. For example, the duty factor of the transducer may be set to between 10% and 100%, between 10% and 90%, between 20% and 80%, between 20% and 30% (e.g., 25% duty factor), between 40% and 90%, between 40% and 60% (e.g., 50% duty factor), between 60% and 80%, between 70% and 80% (e.g., 75% duty factor), any combination of the above-noted ranges, or other values outside of these ranges. In some embodiments, an acoustic transducer operated at a duty factor of 50% or greater may have a tendency to produce liposomes or vesicles incorporating an apolipoprotein that are comparatively more bioactive and/or stable than those produced using a transducer operated at a duty factor of less than 50%.

As an example, when exposing a sample of an apolipoprotein (e.g., ApoA5) to focused acoustic energy, absent a lipid bilayer, in some cases, higher levels of duty factor (e.g., duty factor above 50%) may lead to a comparatively greater degree of degradation of the ApoA5 than focused acoustic energy generated at lower levels of duty factor (e.g., duty factor lower than 50%). Though, when the apolipoprotein is combined with an appropriate lipid formulation and subject to the same overall parameters of focused acoustic energy, with relatively higher duty factors (e.g., duty factors greater than or equal to 50%), a tendency was observed, unexpectedly, for there to be a comparatively reduced amount of damage, if any damage at all, to the apolipoprotein incorporated within the liposome.

Without wishing to be bound by theory, it is thought that the lipid bilayer provides for a suitable degree of absorption of the acoustic energy to occur. The lipid bilayer may further protect the sensitive apolipoprotein contents of the composition, as it becomes incorporated with the lipid bilayer. Accordingly, the combined formulation of apolipoprotein and lipid bilayer may be processed at a duty factor greater than that which the apolipoprotein without the lipid bilayer may be able to withstand without substantial degradation or damage.

In general, as the duty factor of a transducer generating a focused acoustic wavetrain is increased, for some instances, the average size of the vesicles produced decreases. In some cases, such a decrease in vesicle size may be logarithmic.

As an example, a transducer operated at a duty factor of approximately 10% (with PIP=150 W, CPB=1000, 2 mL samples within a vessel kept within a bath maintained at 10° C.) may yield suitably bioactive apolipoprotein carrying liposomes having an average size of between 3.5 microns and 5.0 microns; though, an increase in duty factor to approximately 30%, keeping other parameters the same, may give rise to the formation of apolipoprotein carrying liposomes having a relatively smaller average size, for example, of between 2.5 microns and 4.0 microns; further increase of the duty factor to approximately 50% may result in apolipoprotein carrying liposomes having an even smaller average size, for example, of between 1.0 micron and 2.5 microns. In another example, under the same conditions except the temperature of the bath being maintained at 3° C., while a transducer operated at a duty factor of approximately 10% may yield apolipoprotein carrying liposomes having an average size of between 200 nm and 400 nm, an increase in duty factor to approximately 30% may give rise to apolipoprotein carrying liposomes having an average size of between 150 nm and 300 nm; and an additional increase of the duty factor to approximately 50% may result in apolipoprotein carrying liposomes having an average size of between 100 nm and 250 nm.

As discussed above, an increase in duty factor may result in an overall increase in duration of applied acoustic energy per burst. This provides more time for shear forces to act on the lipid molecules and, hence, form suitably sized and arrangements of phospholipid bilayer fragments. The relaxation time between acoustic bursts gives phospholipid bilayer fragments of a certain size range sufficient time to self-assemble into appropriately configured and sized vesicles (e.g., liposomes). Accordingly, without wishing to be bound by theory, it is thought that phospholipid bilayer fragments larger than the particular size range do not have enough time to form vesicles before the next acoustic burst, and phospholipid bilayer fragments smaller than the size range are unable to fuse together to achieve the necessary size to form the vesicle prior to the next burst. As duty factor increases, the relaxation time decreases, and the particular size range that is able to curl or otherwise assemble into vesicles within that time decreases.

The acoustic transducer may be operated according to a suitable cycles per burst setting to achieve preferred results. As described herein, the cycles per burst (CPB) is the number of acoustic oscillations contained in the active period of one cycle.

In some embodiments, to form a liposome or vesicle that suitably incorporates an apolipoprotein, the acoustic transducer may be operated to generate focused acoustic energy according to a cycles per burst setting of greater than or equal to 50, greater than or equal to 100, greater than or equal to 500, greater than or equal to 1,000, greater than or equal to 1,500, greater than or equal to 2,000, greater than or equal to 2,500, greater than or equal to 3,000, greater than or equal to 3,500, greater than or equal to 4,000, greater than or equal to 4,500, or greater than or equal to 5,000. For example, the transducer may be set to generate focused acoustic energy at a cycles per burst of between 50 and 5,000, between 50 and 1,000, between 500 and 2,000 (e.g., 1,000), between 500 and 1,000, between 1,000 and 5,000, between 4,000 and 6,000 (e.g., 5000), any combination of the above-noted ranges, or other values outside of these ranges.

In some embodiments, focused acoustic energy generated at relatively high levels of cycles per burst (e.g., approximately 1,000, 5,000 cycles per burst) may have a tendency to produce liposomes or vesicles that are smaller and, in some cases, more stable than those produced with acoustic energy generated at a comparatively lower cycles per burst (e.g., approximately 50, 100 cycles per burst).

The cycles per burst of the focused acoustic energy may serve to regulate the size of stable cavitation bubbles, which may aid in liposome formation and, thus, control overall size and polydispersity of the liposomes. In various embodiments, as the cycles per burst of the focused acoustic wavetrain generated by a transducer is increased, the average size of the vesicles produced may decrease, in some cases, logarithmically.

As an example, a transducer operated to generate a focused acoustic wavetrain at a cycles per burst of approximately 50 (with PIP=150 W, DF=50%, 2 mL samples within a vessel kept within a bath maintained at 3° C., processed for 2 minutes) may form suitably bioactive apolipoprotein carrying liposomes having an average size of between 150 nm and 450 nm, though, an increase in the cycles per burst to approximately 500, while maintaining other parameters constant, may give rise to the formation of apolipoprotein carrying liposomes having an average size of between 150 nm and 300 nm. An additional increase of the cycles per burst to approximately 1,000 may result in apolipoprotein carrying liposomes having an average size of between 100 nm and 250 nm. In another example, under the same conditions except the temperature of the bath being maintained at 10° C. and the duration of acoustic treatment lasting for 2 hours, a transducer operated at a cycles per burst of approximately 50 may yield apolipoprotein carrying liposomes having an average size of between 100 nm and 300 nm. An increase in cycles per burst to approximately 500 may give rise to apolipoprotein carrying liposomes having an average size of between 100 nm and 200 nm. Further increase of the cycles per to approximately 1000 may result in apolipoprotein carrying liposomes having an average size of between 100 nm and 150 nm.

It has been observed that as the cycles per burst of the acoustic energy increases, in some cases, there may be a corresponding increase in the radius of the stable cavitation bubbles that generate shear forces that break apart lipids into phospholipid bilayer fragments. With comparatively larger cavitation bubble sizes, more lipid is generally able to come into contact with, or be affected by, the shear forces produced around the bubbles. Accordingly, more lipid can be processed at a time, producing more phospholipid bilayer fragments, under a relatively even distribution. Further, for some instances, relatively large phospholipid bilayer fragments may be broken down more readily than if comparatively smaller cavitation bubbles are produced.

As further discussed herein, the acoustic transducer may be operated so as to produce focused acoustic energy that results in a suitable level of energy input to the sample material. In some embodiments, the transducer may generate acoustic energy having a peak incident power over the course of a period of time that produces a particular amount of energy, to achieve preferred results. As described herein, the peak incident power (PIP) is the power emitted from the transducer during the active period of one cycle. The peak incident power, in some cases, may control the amplitude of the acoustic oscillations. The energy applied to the sample material may be determined by recording the peak incident power of the applied acoustic energy over the duration of the acoustic treatment period.

In some embodiments, to form a liposome or vesicle that suitably incorporates an apolipoprotein, the acoustic transducer may be operated so as to generate focused acoustic energy according to a peak incident power of greater than or equal to 20 Watts, greater than or equal to 40 Watts, greater than or equal to 60 Watts, greater than or equal to 80 Watts, greater than or equal to 100 Watts, greater than or equal to 150 Watts, greater than or equal to 200 Watts, greater than or equal to 250 Watts, greater than or equal to 300 Watts, greater than or equal to 350 Watts, greater than or equal to 400 Watts, greater than or equal to 450 Watts, or greater than or equal to 500 Watts. For example, the peak incident power of the focused acoustic wavetrain may be between 20 Watts and 500 Watts, between 20 Watts and 200 Watts, between 20 Watts and 150 Watts, between 20 Watts and 100 Watts, between 100 Watts and 500 Watts, between 200 Watts and 500 Watts, any combination of the above-noted ranges, or other values outside of these ranges.

In some embodiments, as the transducer is controlled to increase the peak incident power of the applied focused acoustic energy, the average size of the lipid bilayer vesicles (e.g., apolipoprotein carrying liposomes) may be reduced. In some cases, this reduction in average size may be logarithmic. In general, peak incident power may be increased as the amplitude of the acoustic wave is increased, hence, increasing the overall energy imparted to the sample material over a given duration of time.

In an example, a transducer operated to generate a focused acoustic wavetrain at a peak incident power of approximately 50 Watts (with DF=50%, CPB=1,000, and 2 mL samples within a vessel kept within a bath maintained at 3° C.) may yield apolipoprotein carrying liposomes having an average size of between 400 nm and 700 nm, though, an increase in the peak incident power to approximately 100 Watts, keeping other parameters constant, may give rise to the formation of apolipoprotein carrying liposomes having an average size of between 200 nm and 300 nm. An additional increase of the peak incident power to approximately 150-300 Watts may result in apolipoprotein carrying liposomes having an average size of between 100 nm and 200 nm. In another example, under the same conditions except the temperature of the bath being maintained at 10° C., a transducer operated at a peak incident power of approximately 100 Watts may yield apolipoprotein carrying liposomes having an average size of between 250 nm and 400 nm. In some cases, an increase in peak incident power to approximately 150 Watts may give rise to apolipoprotein carrying liposomes having an average size of between 100 nm and 200 nm.

The total amount of energy applied to a sample exposed to focused acoustics may be determined according to the following relationship: Energy=(PIP)*(DF)*(t) where (PIP)=peak incident power, (DF)=duty factor, and (t)=duration of acoustic treatment.

Accordingly, the amount of energy applied to the sample may vary depending on any of the peak incident power, duty factor and duration of acoustic treatment. For example, if the transducer is operated such that the peak incident power of the focused acoustic wavetrain is relatively low (e.g., 20 Watts), the total amount of energy applied to the sample may still be within a suitable range sufficient for acoustic treatment, provided appropriate compensation by the duty factor (e.g., 75%) and/or the duration of acoustic treatment (e.g., 30 minutes). Similarly, as an example, for a short duration of acoustic treatment (e.g., 30 seconds), adjustments may be made to the duty factor and/or the peak incident power (e.g., 500 Watts) so as to provide for a sufficient preferred total amount of energy applied to the sample.

The sample may be exposed to focused acoustic energy for any suitable duration of time. In some embodiments, focused acoustic energy may be applied to sample for at least 10 seconds, at least 20 seconds, at least 30 seconds, at least 60 seconds, at least 120 seconds, at least 180 seconds, at least 240 seconds, at least 300 seconds, at least 10 minutes, at least 15 minutes, at least 20 minutes, at least 25 minutes, at least 30 minutes, at least 45 minutes, or at least 1 hour. The amount of time in which focused acoustic energy is applied to the sample may depend, in part, on the desired total amount of energy to be imparted to the sample. For example, when it is preferred for relatively large amounts of energy to be applied to the sample, longer times of exposure of the sample to focused acoustics may be warranted. Or, when smaller amounts of energy are to be applied to the sample, comparatively shorter times of focused acoustic exposure may occur.

The total amount of energy applied to the sample from the focused acoustic wavetrain may fall within any suitable range. In some embodiments, the total amount of energy applied to the sample may be greater than or equal to 1,000 J, greater than or equal to 2,000 J, greater than or equal to 3,000 J, greater than or equal to 4,000 J, greater than or equal to 5,000 J, greater than or equal to 6,000 J, greater than or equal to 7,000 J, greater than or equal to 8,000 J, greater than or equal to 9,000 J, greater than or equal to 10,000 J, greater than or equal to 12,000 J, or greater than or equal to 14,000 J. For instance, the total amount of energy applied to the sample may be between 1,000 J and 10,000 J, between 2,000 J and 5,000 J, between 5,000 J and 10,000 J, between 7,000 J and 10,000 J, between 10,000 J and 12,000 J, between 12,000 J and 15,000 J, any combination of the above-noted ranges, or other values outside of these ranges.

Similar to that described above with respect to duty factor and peak incident power, for some embodiments, application of increasing amounts of energy to an appropriate lipid formulation for forming lipid bilayer vesicles (e.g., apolipoprotein carrying liposomes) may generally result in the average size of the vesicles to be reduced, as well as tighter distributions (i.e., lower standard deviations). Though, it can be appreciated that for embodiments where apolipoproteins are incorporated with the lipid formulation, it may be preferred for the total amount of energy applied to be high enough such that lipid bilayer liposomes or vesicles incorporating the apolipoproteins are suitably formed, yet, it may be further preferable for the total amount of energy applied to be balanced such that the apolipoproteins remain bioactive. That is, the total amount of energy applied would not be so high that the apolipoproteins degrade and/or lose their therapeutic properties, yet sufficient enough for suitable formation of a liposome or vesicle carrying the apoplipoprotein(s).

In various embodiments, upon delivery of liposomes carrying apolipoprotein in accordance with the present disclosure to cells and surrounding tissue, the formulation is non-toxic, stable and bioactive. For example, delivery of a liposome incorporated with apolipoprotein (e.g., ApoA5) to a cell (e.g., hepatic cell) may result in the apolipoprotein being transferred across the cell wall and, in some cases, for the apolipoprotein to be secreted into the surrounding environment (e.g., plasma), ultimately affecting triglyceride metabolism.

For some embodiments, the following processing parameters may be particularly effective in preparing ApoA5 incorporated with a liposome to remain intact and bioactive. Such parameters may involve a total amount of energy of approximately 5,000 J, at a 100% duty factor, 100 Watts peak incident power, for at least 50 seconds. However, if the duty factor is reduced to 50%, the peak incident power or the duration of acoustic treatment may be doubled, without degrading ApoA5. That is, in this example, the total amount of energy applied to the sample may remain approximately the same despite the peak incident power being raised to 200 Watts, applied for 50 seconds, with the duty factor lowered to 50%. Or, conversely, the total amount of energy applied to the sample may fall within an appropriate range so as to yield desirable results given a peak incident power of 100 Watts, applied for 100 seconds, with a 50% duty factor.

As also discussed above, the mixture including an apolipoprotein and lipid formulation may be processed under appropriate temperature conditions, which may provide additional non-acoustic energy to the mixture. The mixture undergoing focused acoustic processing may be subject to appropriate temperature control, for example, by a water bath, jacket, chiller or other suitable temperature control system. For example, the temperature of the surrounding environment (e.g., water bath in which a vessel holding the sample is located) may be controlled to be between 0° C. and 50° C., between 1° C. and 30° C., between 2° C. and 25° C., between 3° C. and 20° C., between 3° C. and 10° C., or any other suitable temperature that may fall outside of these ranges. In some cases, when the sample is processed under a higher temperature, the total amount of energy required to bring about a desired result may be lower than would otherwise be the case under a lower temperature. Or, the temperature surrounding the sample may be maintained at a relatively low temperature (e.g., approximately 3-4° C.) so that the apolipoprotein remains suitably bioactive.

The mixture of apolipoprotein and lipid formulation may be processed under desirable pressure conditions which, in some cases, may enhance overall efficiency in processing of the mixture. In some embodiments, the mixture is processed using focused acoustics under a positive applied pressure, greater than ambient pressure (e.g., via a decrease in volume, compressive force applied to a treatment area, injection of an inert gas, etc.). For example, the pressure under which the mixture is acoustically processed may be controlled to be between 1 atm and 15 atm, 1 atm and 10 atm, 1 atm and 5 atm, 1 atm and 4 atm, 1 atm and 3 atm, 1 atm and 2 atm, or any other suitable pressure falling outside of the above ranges. In some embodiments, the mixture is processed using focused acoustics under a negative pressure. In some embodiments, to produce a desired result, subjecting the sample to focused acoustic processing under positive pressure may require a comparatively less amount of total acoustic energy input into the system than would otherwise be the case.

The mixture including an apolipoprotein and lipid formulation may have any suitable sample volume. In some embodiments, the sample volume of the mixture processed by focused acoustic energy may be less than 5 mL, less than 3 mL, less than 2 mL, less than 1 mL, less than 500 microliters, less than 200 microliters, or less than 150 microliters (e.g., approximately 130 microliters, 100 microliters), less than 100 microliters (e.g., approximately 72 microliters), less than 50 microliters, less than 10 microliters, less than 5 microliters, or less than 1 microliters. Alternatively, the sample volume of the mixture processed by focused acoustic energy may be greater than or equal to 1 mL. Sample volumes outside of the above-noted ranges are also possible.

In some cases, to achieve a desired result, the total amount of acoustic energy imparted to the mixture may correlate with the sample volume. In some embodiments, for a relatively small sample volume (e.g., approximately 100 microliters), the total amount of acoustic energy applied to yield a suitable composition (e.g., bioactive liposome incorporating apolipoprotein) may vary appropriately (e.g., approximately 2,000-3,000 J at 20 Watts PIP). As an example, subjecting a sample including phosphatidylcholine stabilized with ascorbyl palmitate (e.g., Phospholipon® 90G) incorporating an apolipoprotein (e.g., ApoA5), held within a 72 microliter vessel, to focused acoustic energy having an average power of approximately 75 Watts for about 10 minutes (e.g., applying approximately 2,000-5,000 J) may be preferable to result in a suitable liposome encapsulating the apolipoprotein. Though, for larger sample volumes (e.g., approximately 10 milliliters), to achieve a similar result, it may be preferable for the total amount of acoustic energy imparted to be greater (e.g., approximately 10,000-15,000 J at 500 Watts PIP). Accordingly, subjecting a mixture including an apolipoprotein and lipid formulation to focused acoustic energy to form a suitably bioactive composition may relate, in part, to the energy volume density imparted to the mixture, i.e., the amount of acoustic energy imparted to the sample for a given amount of sample volume.

The transducer may be operated so as to provide an amount of focused acoustic energy that results in a suitable energy volume density imparted to the sample. In some embodiments, the energy volume density imparted to the sample may be between 1,000 J/mL and 10,000 J/mL, between 2,000 J/mL and 8,000 J/mL, between 5,000 J/mL and 10,000 J/mL, between 2,000 J/mL and 4,000 J/mL, between 4,000 J/mL and 6,000 J/mL, between 6,000 J/mL and 8,000 J/mL, or between 8,000 J/mL and 10,000 J/mL. The energy volume density imparted to the sample may vary outside of the above noted ranges.

In accordance with aspects of the present disclosure, subjecting a mixture including an apolipoprotein and lipid formulation to focused acoustic energy operated under certain combinations of acoustic parameters may result in a liposome incorporating an apolipoprotein that is able to provide a preferred therapeutic effect upon delivery to cells or tissue.

In some embodiments, in forming a preferred apolipoprotein carrying liposome formulation, the acoustic transducer may be operated at a duty factor of 25%-100% (e.g., approximately 50%, 75% duty factor), cycles per burst between 50-2,000 (e.g., approximately 1,000 cycles per burst), peak incident power between 10-500 Watts (e.g., approximately 20 Watts, 150 Watts), applying a total amount of energy of between 2,000-9,000 J (e.g., approximately 2,500 J) to the sample mixture (e.g., including a lipid formulation and an apolipoprotein) having a suitable volume (e.g., approximately 1-20 mL), held within a suitable vessel (e.g., 72 microliter volume vessel). The duration of focused acoustic treatment may last for at least 30 seconds (e.g., approximately 30-60 seconds, 10-60 minutes). The temperature immediately surrounding the sample mixture may be maintained between 1-10° C. (e.g., approximately 3° C. or 10° C.) during focused acoustic treatment. The pressure of the environment within which the sample mixture is contained may also be kept to between 1-5 atm during processing. In an example, the transducer may be operated at a duty factor of about 50%, peak incident power of about 150 Watts, cycles per burst of about 1,000, for 10 minutes for acoustically treating a sample having a volume of about 2 mL, and including a mixture of Phospholipon® 90G and ApoA5.

The mixture including the apolipoprotein and initial lipid formulation, processed via focused acoustics, may include additional components, such as an aqueous solution or other additives (e.g., biological or chemical constituent(s), etc.). For example, the mixture may include a surfactant (e.g., sodium dodecyl sulfate) for lowering the interfacial tension between certain molecules of the mixture. In some embodiments, the mixture further includes an aqueous component, for example, water, phosphate buffered saline (e.g., approximately 0.1 M PBS), etc. In some embodiments, the mixture includes a solvent (e.g., acetone, toluene, chloroform, ethanol, etc.), for example, provided for lipid dissolution. Though, it can be appreciated that, for some embodiments, a solvent is not required.

The initial lipid formulation with which the apolipoprotein macromolecules are mixed prior to treatment with focused acoustic energy, for forming an appropriate lipid bilayer, may include any suitable material. In various embodiments, the initial lipid formulation may include phospholipids, lecithin, phosphatidylcholines, phosphatidylcholine stabilized with ascorbyl palmitate (e.g., Phospholipon® 90G), phosphocholine (e.g., 1,2-Distearoyl-sn-glycero-3-phosphocholine), phosphoglycerol (e.g., 1,2-Distearoyl-sn-glycero-3-phosphoglycerol), phosphatidylethanolamines, phosphatidylinositol, or any other appropriate material for preparing liposomes or other lipid bilayer vesicles.

The lipids of the initial lipid formulation within the mixture to be subject to focused acoustic treatment may have any suitable concentration. In some embodiments, the concentration of the one or more lipids (e.g., DSPC, DSPG, etc.) of the initial lipid formulation within the mixture may be between 1 mg/mL and 50 mg/mL, between 1 mg/mL and 10 mg/mL, between 1 mg/mL and 20 mg/mL, between 1 mg/mL and 30 mg/mL, between 1 mg/mL and 40 mg/mL, between 1 mg/mL and 5 mg/mL, between 10 mg/mL and 15 mg/mL, between 15 mg/mL and 20 mg/mL, between 20 mg/mL and 25 mg/mL, between 25 mg/mL and 30 mg/mL, between 35 mg/mL and 40 mg/mL, between 40 mg/mL and 45 mg/mL, or between 45 mg/mL and 50 mg/mL. The concentrations of lipid(s) within the mixture may vary outside of the above noted ranges.

Any appropriate apolipoprotein macromolecules may be included in the initial mixture, for incorporation thereof within and/or throughout a lipid bilayer. As discussed above, apolipoproteins may function as structural components of lipoprotein particles, cofactors for enzymes and/or ligands for cell-surface receptors. When suitably incorporated with the lipid formulation, the apolipoprotein may be suitably packaged so as to provide a therapeutic effect upon delivery to a patient. In some embodiments, the apolipoprotein for incorporation with a lipid bilayer may include apolipoprotein A, apolipoprotein B, apolipoprotein C, apolipoprotein D, apolipoprotein E, apolipoprotein H and apolipoprotein L.

The apolipoprotein within the mixture to be subject to focused acoustic treatment may have any suitable concentration. In some embodiments, the concentration of apolipoprotein (e.g., ApoA5) within the mixture may be between 0.5 micrograms/mL and 50 micrograms/mL, between 0.5 micrograms/mL and 40 micrograms/mL, between 0.5 micrograms/mL and 30 micrograms/mL, between 0.5 micrograms/mL and 20 micrograms/mL, between 0.5 micrograms/mL and 20 micrograms/mL, between 0.5 micrograms/mL and 10 micrograms/mL, between 0.5 micrograms/mL and 5 micrograms/mL, between 0.5 micrograms/mL and 2.0 micrograms/mL, between 2.0 micrograms/mL and 8.0 micrograms/mL, between 8.0 micrograms/mL and 15.0 micrograms/mL, between 15.0 micrograms/mL and 20.0 micrograms/mL, between 20.0 micrograms/mL and 30.0 micrograms/mL, between 30.0 micrograms/mL and 40.0 micrograms/mL, or between 40.0 micrograms/mL and 50.0 micrograms/mL. The concentrations of apolipoprotein(s) within the mixture may vary outside of the above noted ranges.

Apolipoprotein A may include at least one of apolipoprotein A-I, apolipoprotein A-II and apolipoprotein A-IV and apolipoprotein A-V, each of which may have therapeutic effects when appropriately administered via a liposome or other lipid bilayer. Apolipoprotein A-I is a major protein component in high-density lipoprotein (HDL) particles in plasma that promotes fat efflux, including cholesterol, from tissues to the liver for excretion. Further, apolipoprotein A-I is a cofactor for lecithin cholesterolacyltransferase, which is an enzyme used for the formation of a number of plasma cholesteryl esters, and has been known to exhibit anticlotting effects. Apolipoprotein A-II is also a protein component in HDL particles. Apolipoprotein A-IV has been linked to intestinal lipid absorption. As discussed above, apolipoprotein A-V is a determinant of triglyceride levels in plasma and may be used therapeutically to enhance lipoprotein metabolism.

Apolipoprotein B may include at least one of apolipoprotein B48 and apolipoprotein B100, each of which may also be therapeutically administered. Apolipoprotein B are the primary apolipoproteins of chylomicrons and low-density lipoproteins (LDL), which are responsible for carrying cholesterol to tissues and cells. High levels of apolipoprotein B have been correlated with plaques that cause vascular disease (atherosclerosis). A rare autosomal recessive disorder called abetalipoproteinaemia, which interferes with the normal absorption of fat and fat-soluble vitamins, is caused by a deficiency of apolipoprotein B48 and apolipoprotein B100.

Apolipoprotein C may include at least one of apolipoprotein C-I, apolipoprotein C-II, apolipoprotein C-III and apolipoprotein C-IV. Each of the above molecules may also be therapeutically administered via a lipid bilayer vesicle such as a liposome. Apolipoprotein C-I, C-II, C-III and C-IV are surface components of chylomicrons, VLDL, and HDL. Apolipoprotein C-I is a positively charged protein component of lipoproteins that is normally found in plasma. This protein is responsible for the activation of esterified lecithin cholesterol, leading to the exchange of esterified cholesterol between lipoproteins, and in removal of cholesterol from tissues. Apolipoprotein C-II is a protein component of very low density lipoproteins (VLDL) and chylomicrons. This protein activates lipoprotein lipase in capillaries, which hydrolyzes triglycerides so as to liberate fatty acids and monoglycerides to surrounding tissue and/or cells (e.g., adipocytes, muscle). Deficiencies in apolipoprotein C-II may lead to an accumulation of chylomicrons and triglycerides. Apolipoprotein C-III is also a component of VLDL, and may promote the assembly and secretion of triglyceride-rich VLDL particles from hepatic cells under certain conditions (e.g., lipid-rich conditions). Apolipoprotein C-III may function to inhibit lipoprotein lipase and hepatic lipase. Apolipoprotein C-IV is a protein that is formed in the liver and has a protein structure similar to other apolipoprotein C molecules.

Apolipoprotein D is a protein component of high density lipoprotein. Apolipoprotein D is often used as a biomarker for androgen insensitivity syndrome.

Apolipoprotein E is found in chylomicrons and intermediate-density lipoproteins (IDL), which are required for normal catabolism of triglyceride-rich lipoprotein constituents. Apolipoprotein E is often produced by the liver and macrophages, and mediates cholesterol metabolism. Apolipoprotein E may also be produced by astrocytes in the central nervous system, for transporting cholesterol to neurons. Apolipoprotein E is also found in blood plasma, mediating transport and cellular uptake of cholesterol and lipids.

Apolipoprotein H is a protein that is thought to be involved in a number of physiological processes. Such processes include blood coagulation, haemostasis and the production of antiphospholipid antibodies characteristic of antiphospholipid syndrome.

Apolipoprotein L is a high density lipoprotein and plays a role in mediating cholesterol transport. Regulation of the cholesterol content of membranes is important for certain cellular processes, such as gene transcription modulation and signal transduction, both in the adult brain and during neurodevelopment.

Liposomes described herein may be produced so as to have a number of suitable characteristics. For example, as discussed above, the liposomes may at least partially encapsulate or otherwise incorporate an apolipoprotein therewith. The apolipoprotein may be packaged with the liposome in a way that allows for a desired therapeutic effect to take place upon delivery of the liposome carrying apolipoprotein to a patient (e.g., via injection, ingestion, surgical placement, etc.). In various embodiments, particular combinations of focused acoustic parameters may be employed so as to produce a liposome carrying apolipoprotein consistently and reliably.

In various embodiments, the size of liposomes that incorporate apolipoprotein in accordance with aspects of the present disclosure may be controlled based on the appropriate acoustic processing parameters employed. For instance, an increase in one or more of peak incident power, duty factor, cycles per burst, temperature, pressure, or other parameters may, in some cases, as discussed above, lead to a general reduction in size of the liposomes produced. It may be preferable for various embodiments of liposome formulations to be small, having a relatively high surface area, which may give rise to a heightened degree of bioavailability.

In some embodiments, the average size of the acoustically formed liposomes/vesicles carrying apolipoprotein may be less than 1.0 micron, less than 800 nm, less than 600 nm, less than 500 nm, less than 400 nm, less than 300 nm, less than 200 nm, less than 100 nm, less than 50 nm, less than 30 nm, less than 10 nm, or less than 5.0 nm. For example, liposomes in accordance with some embodiments may have an average size of between 5 nm and 800 nm, between 5 nm and 200 nm, between 10 nm and 500 nm, between 10 nm and 30 nm (e.g., approximately 20 nm, approximately 25 nm), between 50 nm and 500 nm, between 50 nm and 150 nm (e.g., approximately 100 nm), between 150 nm and 250 nm (e.g., approximately 200 nm), any combination of the above-noted ranges, or other values outside of these ranges. The average size as provided herein is measured by dynamic laser light scattering, i.e., photon correlation spectroscopy, using a Malvern Zetasizer Nano ZS-90 instrument, at 25° C. Each sample is diluted with distilled water immediately before measurement, to avoid multiple light scattering effects.

Apolipoprotein carrying liposomes, or other liposomes or vesicles, formed via focused acoustics may exhibit a suitable particle size distribution. In some embodiments, the standard deviation of the particle size distribution is less than 500 nm, less than 400 nm, less than 300 nm, less than 200 nm, less than 150 nm, less than 100 nm, less than 50 nm, less than 30 nm, less than 20 nm, less than 10 nm, less than 5 nm, or less than 1 nm. The standard deviation of the particle size distribution of such liposomes or vesicles may vary outside of these ranges. In some cases, it may be preferable for the particle size distribution of the liposomes/vesicles to be relatively tight, or have a low standard deviation.

Focused acoustic treatment processes may be scaled up to acoustically treat any appropriate volume of sample material in accordance with systems and methods provided herein. In some embodiments, a treatment vessel may have one or more suitable inlets and/or outlets that permit sample material to flow into and out of the vessel or a process chamber of the vessel. Once suitably disposed in the vessel or process chamber, the sample material may be subject to focused acoustic treatment under an appropriate set of conditions. After a sufficient degree of focused acoustic treatment, the sample material may be discharged from the vessel or process chamber, allowing more sample that had not been previously treated to be subject to focused acoustic treatment. For various embodiments described herein, a treatment vessel may be considered to be equivalent to a process chamber.

In some embodiments, an acoustic treatment system may include a reservoir and a process chamber, each having inlets and outlets that are in fluid communication with one another; that is, fluid is permitted to travel between the reservoir and the process chamber via suitable conduits. Accordingly, sample material from the reservoir may be caused to travel to the process chamber for focused acoustic treatment under appropriate conditions and may subsequently be caused to travel back to the reservoir. As a result, sample material may be acoustically processed in a cyclic fashion where portions of sample material may receive focused acoustic treatment multiple times.

In some embodiments, sample material may travel from a supply reservoir to a process chamber for focused acoustic treatment. The treated sample material may subsequently travel from the process chamber to a different container separate from the supply reservoir. As such, the sample material may undergo a single acoustic treatment.

In some embodiments, sample material may travel from a supply reservoir through multiple process chambers for varying levels of processing, such as different conditions of focused acoustics. Additional conduits may also be provided for the addition/removal of sample material, which may serve to enhance encapsulation or incorporation of apolipoprotein with the liposome or other lipid bilayer. In an example, additional material may be introduced into the sample through a conduit and, upon combination with the sample material, packaging of the apolipoprotein with the lipid bilayer may be enhanced. Accordingly, continuous flow manufacturing of apolipoprotein suitably incorporated with a lipid bilayer may be achieved.

### Example

The following example is intended to be illustrative of certain embodiments of the present disclosure, and is not to be construed as limiting and does not exemplify the full scope of the present disclosure.

In this example, focused acoustic energy was used to incorporate ApoA5 within liposomes. Here, 32 mg of 1,2-Distearoyl-sn-glycero-3-phosphocholine (DSPC) and 8 mg of 1,2-Distearoyl-sn-glycero-3-phosphoglycerol (DSPG) were added to a 2 mL aqueous solution of 0.1 M phosphate buffered saline, held within a 130 micron glass vessel. ApoA5 was then added to the mixture at a concentration of 5 micrograms/mL.

The mixture was then subject to focused acoustic energy using a S220X Covaris AFA instrument having a transducer with a concave shape, resulting in convergence of acoustic energy to a precise point inside a processing vessel, and providing a total amount of acoustic energy ranging between 2,000 and 14,400 J. For a 72 microliter sample mixture, the transducer was operated to generate acoustic energy having a frequency of approximately 500 kHz, at a peak incident power of 80 Watts, a duty factor of 50% and 1,000 cycles per burst for 10 minutes. For a 2 mL sample mixture, the transducer was operated to generate acoustic energy having a frequency of approximately 500 kHz, at a peak incident power of 150 Watts, a duty factor of 50% and 1,000 cycles per burst for 60 minutes. Under the above parameters, liposomes incorporating ApoA5 were produced, having an average size of approximately 104 nm.

The liposomes incorporating ApoA5 were incubated with a human hepatocellular cell line (HepG2) for 24, 48 and 72 hours, and the relative amounts of intracellular and secreted ApoA5 were measured. The cells incubated with liposomes incorporating ApoA5 were compared with cells incubated with ApoA5 (66 ng) without liposomes; cells incubated with liposomes without ApoA5; and cells incubated without ApoA5 or liposomes.

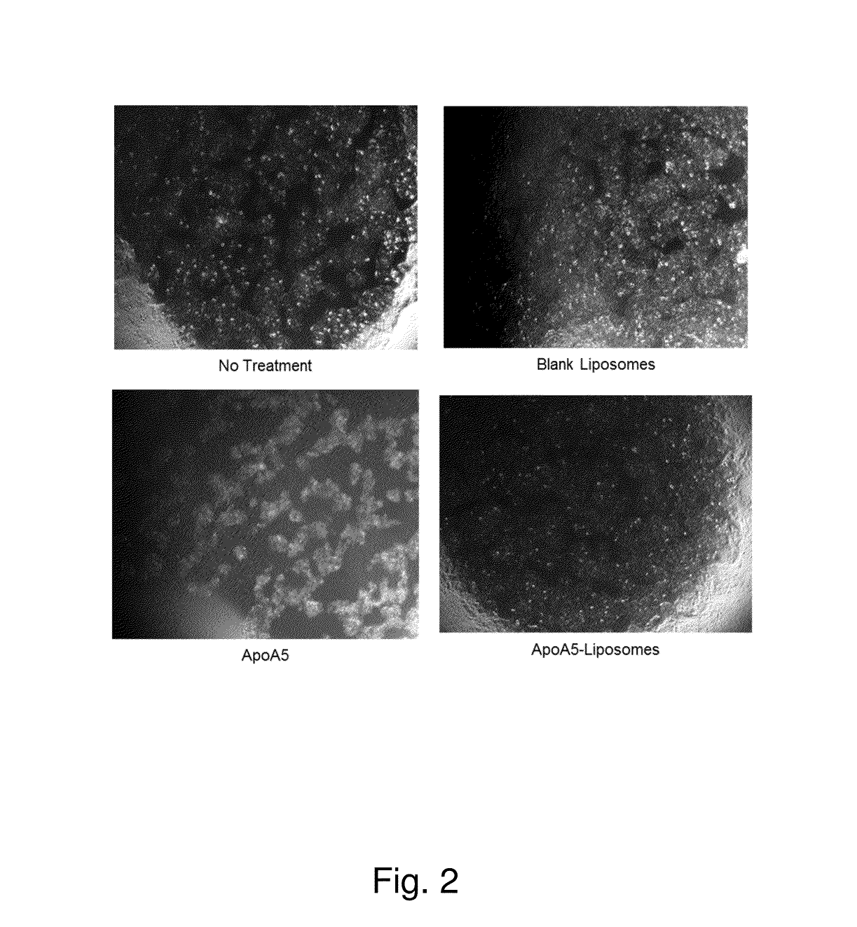

As shown in FIG. 2, incubation of the cells with ApoA5 without liposomes (shown in the bottom left of FIG. 2) resulted in cellular toxicity after 24-48 hours. Cellular toxicity was determined through visual inspection and a cell proliferation assay, both performed via microscopy by a skilled operator. However, treatment of cells with liposomes incorporating ApoA5 (shown in bottom right of FIG. 2) was not observed to be toxic to the cells. After a 48-hour incubation, 40-97% of the ApoA5 (63.79±24.06 ng/mL) incorporated into the liposomes was subsequently introduced into the HepG2 cells. This observation indicated a resulting transport of the ApoA5 across the cell wall, in contrast with the compared cases noted above. ApoA5 transfer across the cell wall was determined via cell lysates that were collected and analyzed using an ApoA5 Elisa Kit provided by Novatein Biosciences.

It is to be understood that the present disclosure is not limited in its application to the details of construction and the arrangement of components set forth in the following description or illustrated in the drawings. Other embodiments and manners of carrying out the present disclosure are possible. The phraseology and terminology used herein is for the purpose of description and should not be regarded as limiting. The use of “including,” “comprising,” or “having” and variations thereof is meant to encompass the items listed thereafter and equivalents thereof as well as additional items.

Having thus described various illustrative embodiments and aspects thereof, modifications and alterations may be apparent to those of skill in the art. Such modifications and alterations are intended to be included in this disclosure, which is for the purpose of illustration only, and is not intended to be limiting. The scope of the present disclosure should be determined from proper construction of the appended claims, and their equivalents.

What is claimed is:

1. A method of preparing a lipid composition, comprising:
providing a mixture including an apolipoprotein and a lipid formulation in a vessel;
transmitting focused acoustic energy through a wall of the vessel such that at least a portion of the mixture is exposed to acoustic energy having a frequency of between about 100 kilohertz and about 100 megahertz at a focal zone having a size dimension of less than about 2 centimeters; and
forming a liposome at least partially encapsulating the apolipoprotein by, at least in part, exposure of the mixture to the focal zone.

2. The method of claim 1, wherein a particle size of the liposome is between 10 nm and 500 nm.

3. The method of claim 1, wherein the liposome fully encapsulates the apolipoprotein.

4. The method of claim 1, wherein the apolipoprotein is integrated into a lipid bilayer of the liposome.

5. The method of claim 1, wherein transmitting focused acoustic energy includes operating an acoustic transducer at a duty factor of greater than or equal to 25%, or between 40% and 90%.

6. The method of claim 1, wherein transmitting focused acoustic energy includes operating an acoustic transducer at a cycles per burst of greater than or equal to 50, between 50 and 1000, between 500 and 1,000, or between 1,000 and 5,000.

7. The method of claim 1, wherein transmitting focused acoustic energy includes operating an acoustic transducer at a peak incident power of greater than or equal to 20 Watts, or between 20 Watts and 500 Watts.

8. The method of claim 1, wherein the mixture has a volume of less than 5 mL, or less than 100 microliters.

9. The method of claim 1, wherein transmitting focused acoustic energy includes operating an acoustic transducer such that an energy volume density applied to the mixture is between 1,000 J/mL and 10,000 J/mL.

10. The method of claim 1, wherein transmitting focused acoustic energy includes operating an acoustic transducer such that a total amount of acoustic energy applied to the mixture is greater than or equal to 2,000 J, greater than or equal to 7,500 J, or between 2,000 J and 20,000 J.

11. The method of claim 1, wherein transmitting focused acoustic energy to expose the mixture to the focal zone occurs for at least 30 seconds, at least 120 seconds, at least 10 minutes, or at least 30 minutes.

12. The method of claim 1, further comprising maintaining a temperature around the mixture to be between 0° C. and 50° C. during exposure to the focused acoustic energy.

13. The method of claim 1, further comprising maintaining a pressure around the mixture to be between 1 atm and 5 atm during exposure to the focused acoustic energy.

14. The method of claim 1, wherein the liposome at least partially encapsulating the apolipoprotein facilitates transfer of the apolipoprotein across a cell wall.

15. The method of claim 1, wherein the mixture exposed to the focused acoustic energy has a volume of between 1 microliter and 1 milliliter.

16. The method of claim 1, further comprising flowing the mixture through an inlet into the vessel and flowing the liposome at least partially encapsulating the apolipoprotein through an outlet out of the vessel.

17. The method of claim 1, wherein the lipid formulation comprises at least one of 1,2-Distearoyl-sn-glycero-3-phosphocholine and 1,2-Distearoyl-sn-glycero-3-phosphoglycerol.

18. The method of claim 1, wherein the mixture includes a solvent.

19. The method of claim 1, further comprising adding a surfactant to the mixture.

20. The method of claim 1, wherein the apolipoprotein is at least one of apolipoprotein A, apolipoprotein B, apolipoprotein C, apolipoprotein D, apolipoprotein E, apolipoprotein H and apolipoprotein L.

21. The method of claim 20, wherein the apolipoprotein A is at least one of apolipoprotein A-I, apolipoprotein A-II and apolipoprotein A-IV and apolipoprotein A-V.

22. The method of claim 20, wherein the apolipoprotein B is at least one of apolipoprotein B48 and apolipoprotein B100.

23. The method of claim 20, wherein the apolipoprotein C is at least one of apolipoprotein C-I, apolipoprotein C-II, apolipoprotein C-III and apolipoprotein C-IV.

24. An apolipoprotein composition, comprising:
a protein including apolipoprotein A-V; and
a lipid bilayer at least partially encapsulating the protein.

25. The composition of claim 24, wherein the lipid bilayer comprises a liposome.

26. The composition of claim 24, wherein the lipid bilayer includes a cavity that contains the protein.

27. The composition of claim 24, wherein the lipid bilayer fully encapsulates the protein.

28. The composition of claim 24, wherein the protein is at least partially integrated within the lipid bilayer.

29. The composition of claim 24, wherein the lipid bilayer includes at least one of DSPC and DSPG.
